(19) United States
(12) Patent Application Publication
(10) Pub. No.: US 2007/0191318 A1
(43) Pub. Date: Aug. 16, 2007

(54) Process for the preparation of amorphous rosuvastatin calcium

(76) Inventors: Yatendra Kumar, Haryana (IN); Mohammad Rafeeq, Haryana (IN); Shantanu De, Delhi (IN); Swargam Sathyanarayana, Pradesh (IN)

Correspondence Address: RANBAXY INC., 600 COLLEGE ROAD EAST, SUITE 2100, PRINCETON, NJ, 08540, US

(21) Appl. No.: 10/576,410

(22) PCT Filed: Oct. 22, 2004
(86) PCT No.: PCT/IB04/03487

§ 371 Date: Feb. 20, 2007

(30) Foreign Application Priority Data
Oct. 22, 2003 (IN) 1304/DEL/2003

(51) Int. Cl. A61K 31/56; A01N 45/00

(52) U.S. Cl. 514/169

(57) ABSTRACT

The invention relates to processes for the preparation of amorphous rosuvastatin calcium. More particularly, it relates to the preparation of pure amorphous rosuvastain calcium and pharmaceutical compositions that include the pure amorphous rosuvastatin calcium. The invention also relates to use of said compositions for treating hyperlipidemia, hypercholesterolemia, and atherosclerosis. Formula (I).

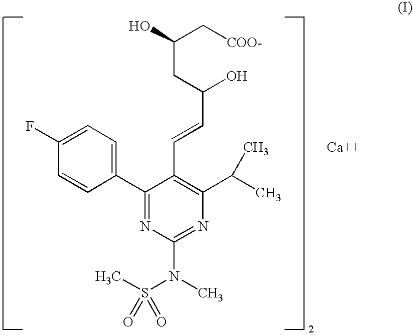

### FIELD OF THE INVENTION

The field of the invention relates to processes for the preparation of amorphous rosuvastatin calcium. More particularly, it relates to the preparation of pure amorphous rosuvastain calcium and pharmaceutical compositions that include the pure amorphous rosuvastatin calcium. The invention also relates to use of said compositions for treating hyperlipidemia, hypercholesterolemia, and atherosclerosis.

### BACKGROUND OF THE INVENTION

Chemically, rosuvastatin calcium is, (3R,5S,6E)-7-[4-(4-fluorophenyl)-6-(1-methylethyl)-2-[methyl(methylsulfonyl)amino]-5-pyrimidinyl]-3,5-dihydroxy-6-heptenoic acid, calcium salt (2:1) having the structural Formula I. It is an antihypercholesterolemic drug used in the treatment of atherosclerosis.

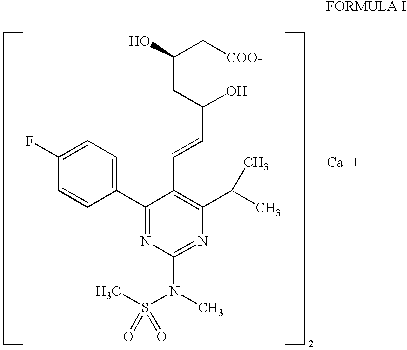

U.S. Pat. No. RE37314 discloses a process for the preparation of amorphous rosuvastatin calcium, which involves dissolving rosuvastatin sodium salt in water, and adding calcium chloride.

U.S. Pat. No. 6,589,959 discloses a process for the preparation of crystalline Form A of rosuvastatin by warming the amorphous form of rosuvastatin calcium in a mixture of water and acetonitrile, and cooling the resultant solution.

The prior art approach for the preparation of amorphous rosuvastatin is not suitable from commercial point of view because the amorphous product is difficult to isolate and the product is not obtained in high purity, thus making the approach commercially difficult to implement. The purity hereto refers to the compound purity, as well as diastereomeric purity. The unwanted diastereomeric impurity is more than 1%.

To achieve a high efficiency of reaction for industrial scale synthesis of amorphous rosuvastain, it is necessary to minimize the formation of the diastereomeric impurity.

Thus, the present invention provides a process which does not result in impure amorphous form; rather pure amorphous form having diastereomeric impurity less than 0.5% is obtained. The amorphous rosuvastatin calcium when made by the process of the present invention is easy to isolate and handle thus making the process amenable for commercial scale use.

### SUMMARY OF THE INVENTION

In one general aspect there is provided a pure amorphous form of rosuvastatin calcium of Formula I having a purity of more than 99% with diastereomeric impurity less than 0.5% by HPLC.

The amorphous form of rosuvastatin calcium may have, for example, the X-ray powder diffraction pattern of FIG. 1.

In another general aspect there is provided a pharmaceutical composition that includes a therapeutically effective amount of the pure amorphous rosuvastatin calcium; and one or more pharmaceutically acceptable carriers, excipients or diluents.

In another general aspect there is provided a process for the preparation of pure amorphous form of rosuvastatin calcium. The process includes obtaining a solution of rosuvastatin calcium in one or more solvents; and recovering the pure amorphous form of rosuvastatin calcium by the removal of the solvent.

The solvent may be, for example, one or more of lower alkanols, ethers, esters, ketones, polar aprotic solvents, water, or mixtures thereof. The lower alkanol may include one or more of primary, secondary and tertiary alcohol having from one to six carbon atoms. The lower alkanol may include one or more of methanol, ethanol, n-propanol, and isopropanol.

The ketone may include one or more of acetone, ethyl methyl ketone, methyl isobutyl ketone, and diisobutyl ketone.

The ester may include one or more of ethyl formate, methyl acetate, ethyl acetate, isopropyl acetate, n-propyl acetate, isobutyl acetate, butyl acetate, and amyl acetate. Examples of ether include tetrahydrofuran and 1,4-dioxane.

The polar aprotic solvent may include one or more of N,N-dimethylformamide, N,N-dimethylacetamide, dimethylsulphoxide, acetonitrile, and N-methylpyrrolidone.

Removing the solvent may include, for example, one or more of distillation, distillation under vacuum, evaporation, spray drying, freeze-drying, lyophilization, filtration, filtration under vacuum, decantation and centrifugation.

The rosuvastatin calcium in an amorphous form may be recovered from the solution by spray drying. Alternatively, the rosuvastatin calcium in an amorphous form may be recovered from the solution by freeze-drying. The process may include further forming of the product so obtained into a finished dosage form.

The amorphous form of rosuvastatin calcium can also be recovered from the solution by adding a suitable additional solvent/second solvent resulting in the precipitation of the amorphous form and removing the solvent there from by filtration, filtration under vacuum, decantation or centrifugation.

The additional solvent/second solvent may be selected from a group of organic solvents in which rosuvastatin calcium is insoluble or poorly soluble or practically insoluble or partially soluble and is known to a person of ordinary skills in the art.

The additional/second solvent may be one or more of isopropanol, isobutanol, n-butanol, cyclopentane, cyclohexane, cycloheptane, hexane, petroleum ether, heptane, diethyl ether, diisopropyl ether, water, or mixtures thereof.

The process may include further drying of the product obtained.

In one general aspect, the solution of rosuvastatin calcium may be obtained by heating the solvent containing rosuvastatin calcium. It may be heated from about 40° C. to about 200° C., for example from about 500° C. to about 150° C. It may be heated from about 10 minutes to about 24 hours.

In another general aspect, the solution may be cooled before filtration to obtain better yields of the pure amorphous form of rosuvastatin calcium.

The process may produce the pure amorphous form of rosuvastatin calcium having a purity of more than 99% with diastereomeric impurity less than 0.5% by HPLC. In particular, it may produce the pure rosuvastatin calcium having a purity of more than 99.5% with diastereomeric impurity less than 0.25%, for example a purity of more than 99.8% with diastereomeric impurity less than 0.15%.

In another general aspect there is provided a process for the preparation of pure/amorphous form of rosuvastatin calcium. The process includes subjecting crystalline rosuvastatin calcium to milling until said crystalline form is converted to the amorphous form.

The crystalline form of rosuvastatin calcium may be, for example in solid state or its slurry in a solvent.

The solvent may be one or more of isopropanol, isobutanol n-butanol, cyclopentane, cyclohexane, cycloheptane, hexane, petroleum ether, heptane, diethyl ether, diisopropyl ether, or mixtures thereof.

The process may include further drying of the product obtained.

In another general aspect there is provided a process for the preparation of the pure amorphous form of rosuvastatin calcium. The process involves lactonizing rosuvastatin methyl ammonium salt of Formula II,

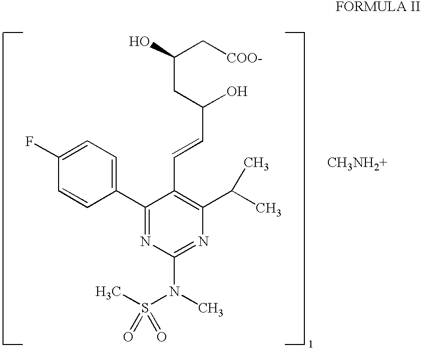

to obtain rosuvastatin lactone of Formula II,

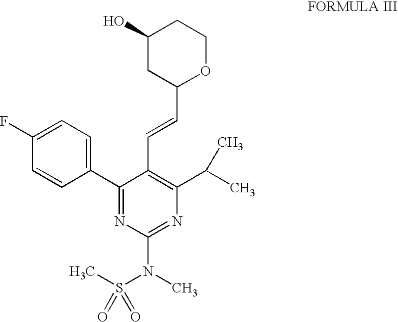

reacting the rosuvastatin lactone with a base and a calcium salt, and recovering the amorphous form of rosuvastatin calcium.

The lactonization may be carried out in presence of an acid in a solvent.

Examples of acid which can be used in the reaction include inorganic acids such as hydrochloric acid, sulfuric acid, nitric acid, phosphoric acid, or organic acids such as formic acid, acetic acid, and the like, or mixtures thereof.

The solvent may be one or more of toluene, xylene, benzene, ethyl methyl ketone, diisobutyl ketone, methyl isobutyl ketone, methyl t-butyl ether, diisopropyl ether, ethyl acetate, methyl formate, methyl acetate, isobutyl acetate, n-propyl acetate, isopropyl acetate, amyl acetate, or mixtures thereof.

The rosuvastatin lactone may be treated with a base and a calcium salt. Examples of such bases include sodium hydroxide, sodium carbonate, sodium bicarbonate, potassium hydroxide, potassium carbonate, and potassium bicarbonate.

The calcium ions may be generated by using a calcium salt. Examples of such calcium salts include calcium chloride, calcium hydroxide, calcium carbonate, calcium acetate, calcium sulphate, calcium borate, calcium tartarate, calcium bromide, or any other compound capable of generating calcium ions.

In one general aspect water may be removed from reaction mass by azeotropic distillation after reacting the rosuvastatin lactone with a base and calcium salt.

In another general aspect there is provided a process for the preparation of pure amorphous form of rosuvastatin calcium. The process involves treating rosuvastatin methyl ammonium salt with a base and a calcium salt; and recovering the amorphous form of rosuvastatin calcium.

Examples of bases which can be used in the reaction include sodium hydroxide, sodium carbonate, sodium bicarbonate, potassium hydroxide, potassium carbonate, and potassium bicarbonate. Examples of calcium salts include calcium chloride, calcium hydroxide, calcium carbonate, calcium acetate, calcium sulphate, calcium borate, calcium tartarate, and calcium bromide.

In another general aspect there is provided a process for the preparation of pure amorphous form of rosuvastatin calcium. The process involves treating rosuvastatin calcium with an acid to obtain rosuvastatin; and converting rosuvastatin to the amorphous form of rosuvastatin calcium by treatment with a base and calcium salt.

The acid may be one or more of inorganic acids such as hydrochloric acid, sulphuric acid, phosphoric acid, hydrobromic acid, nitric acid, or mixtures thereof or organic acids such as formic acid, acetic acid, propionic acid, methanesulphonic acid, 4-toluenesulphonic acid, or mixtures thereof.

The process may include further drying of the product obtained.

The process may produce the amorphous form of the rosuvastatin calcium having the X-ray diffraction pattern of FIG. 1.

The details of one or more embodiments of the inventions are set forth in the description below. Other features, objects and advantages of the inventions will be apparent from the description and claims.

### DESCRIPTION OF THE DRAWINGS

FIG. 1 is an X-ray powder diffraction pattern of amorphous form of rosuvastatin calcium.

### DETAILED DESCRIPTION OF THE INVENTION

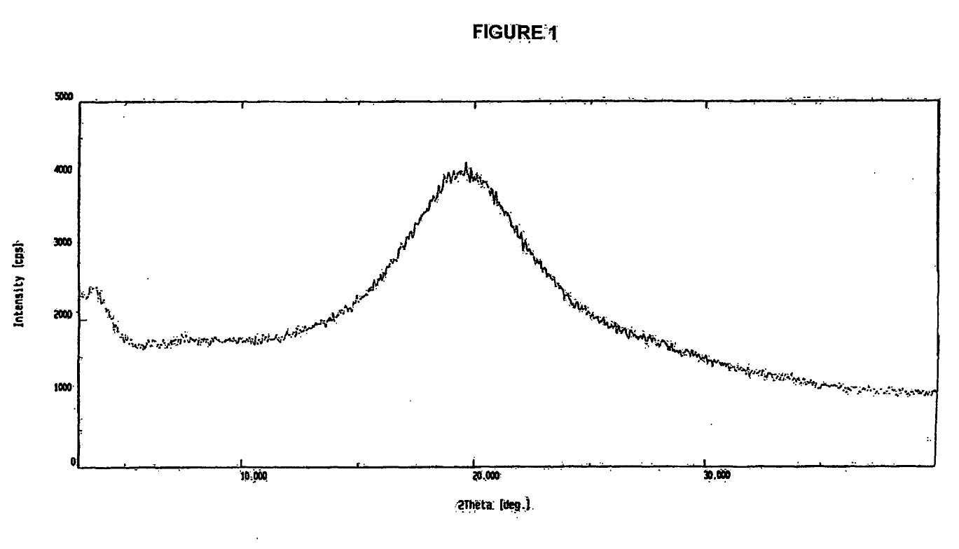

The inventors have developed processes for the preparation of the pure amorphous form of rosuvastatin calcium having a purity of more than 99% with diastereomeric impurity less than 0.5% by HPLC. The pure amorphous form is characterized by its X-ray powder diffraction pattern as shown in FIG. 1. The inventors have developed a process for the preparation of the pure amorphous form of rosuvastatin calcium, by obtaining a solution of rosuvastatin calcium in one or more of solvents; and recovering the amorphous form of rosuvastatin calcium by the removal of the solvent. The inventors also have developed pharmaceutical compositions that contain the amorphous form of the pure rosuvastatin calcium, in admixture with one or more solid or liquid pharmaceutical diluents, carriers, and/or excipients.

In general, the solution of rosuvastatin calcium may be obtained by dissolving crystalline rosuvastatin calcium in a suitable solvent. Alternatively, such a solution may be obtained directly from a reaction in which rosuvastatin calcium is formed. The solution of crystalline rosuvastatin calcium may be obtained by heating the solvent containing crystalline rosuvastatin calcium. It may be heated from about 40° C. to about 200° C., for example from about 50° C. to about 150° C. It may be heated from about 10 minutes to about 24 hours. More particularly, it may be heated for about 2-3 hours. The solution may be filtered to remove any undissolved foreign particulate matter.

The crystalline rosuvastatin calcium can be prepared by methods described in the U.S. Pat. No. 6,589,959.

The term “crystalline rosuvastatin calcium” includes all polymorphic forms, amorphous form, solvates, hydrates, or mixtures thereof.

The solvent may be removed from the solution by a technique which includes, for example, distillation, distillation under vacuum, evaporation, spray drying, freeze-drying, lyophilization, filtration, filtration under vacuum, decantation and centrifugation.

In one aspect, the solution may be concentrated to remove the solvent. The concentration can be carried out under vacuum of about 100 to 0.01 mm of Hg. The solvent may be removed by vacuum distillation of the solution with simultaneous heating the solution at a temperature of about 15 to 55° C. to effect faster removal of the solvent.

In another aspect, rosuvastatin calcium in amorphous form is recovered from the solution using a spray drying technique. A Mini-Spray Dryer (Model: Buchi 190, Switzerland) can be used. The Buchi 190 Mini-Spray Dryer operates on the principle of nozzle spraying in a parallel flow, i.e., the sprayed product and the drying gas flow in the same direction. The drying gas can be air or inert gases such as nitrogen, argon and carbon dioxide. In particular, the drying gas can be nitrogen.

In another aspect, rosuvastatin calcium in amorphous form can be recovered from the solution using a freeze drying technique. A freeze dryer (Model: Virtis Genesis SQ Freeze Dryer) can be used in this technique. The Virtis Genesis SQ Freeze Dryer operates on the principle of lyophilization, i.e., a process of stabilizing initially wet materials (aqueous solution or suspensions) by freezing them, then subliming the ice while simultaneously desorbing some of the bound moisture (primary drying). Following removal of the ice, desorption may be continued (secondary drying). This process may be carried out under vacuum.

The term “suitable solvent” includes any solvent or solvent mixture in which rosuvastatin calcium, is soluble, including, for example, lower alkanol, ketones, esters, ethers, polar aprotic solvents, water, and mixtures thereof. Examples of alkanol include those primary, secondary and tertiary alcohols having from one to six carbon atoms. Suitable lower alkanol solvents include methanol, ethanol, n-propanol, and isopropanol. Examples of ketones include solvents such as acetone, ethyl methyl ketone, methyl isobutyl ketone and diisobutyl ketone. Examples of esters include solvents such as ethyl formate, methyl acetate, ethyl acetate, isopropyl acetate, n-propyl acetate, isobutyl acetate, butyl acetate and amyl acetate. Examples of ethers include tetrahydrofuran and 1,4 dioxane. A suitable polar aprotic solvent includes one or more of N,N-dimethylformamide, N,N-dimethylacetamide, dimethylsulphoxide, acetonitrile and N-methylpyrrolidone. Mixtures of all of these solvents are also contemplated.

In another aspect, a suitable additional/second solvent can be added to the clear solution to precipitate the amorphous form of rosuvastatin calcium. The term “additional/second solvent” includes any solvent in which rosuvastatin calcium is insoluble or poorly soluble or practically insoluble or partially soluble, including, for example, isopropanol, isobutanol, n-butanol, cyclopentane, cyclohexane, cycloheptane, hexane, petroleum ether, heptane, diethyl ether, diisopropyl ether, water, or mixtures thereof.

The product obtained may be further or additionally dried to achieve the desired moisture values. For example, the product may be further or additionally dried in a tray drier, dried under vacuum and/or in a Fluid Bed Dryer.

The inventors have developed a process for the preparation of the pure amorphous form of rosuvastatin calcium, by subjecting the crystalline rosuvastatin calcium to milling until the crystalline form is converted to amorphous form

In general, the crystalline form of rosuvastatin calcium in solid state may be subjected to milling. Alternatively, slurry of rosuvastatin calcium in a solvent may be milled.

In general, the milling involves grinding action between two surfaces. Milling may be carried out using a traditional technique of compounding using a pestle and mortar or by milling machines that essentially work on the same principle. Examples of such milling machines include various makes of ball mills, roller mills, gyratory mills, and the like. The slurry of the crystalline form in a solvent can be from about 30% to 85% w/v.

The term “solvent” includes any solvent or solvent mixture in which rosuvastatin calcium, is insoluble or very slightly soluble or sparingly soluble, including, for example, isopropanol, isobutanol, n-butanol, cyclopentane, cyclohexane, cycloheptane, hexane, petroleum ether, heptane, diethyl ether, diisopropyl ether, or mixtures thereof.

The inventors also have developed a process for the preparation of pure amorphous rosuvastatin calcium, by lactonizing rosuvastatin methyl ammonium salt of Formula II, to obtain rosuvastatin lactone of Formula III; reacting the rosuvastatin lactone with a base and a calcium salt; and recovering the amorphous rosuvastatin calcium.

The rosuvastatin methyl ammonium salt may be prepared by the methods described in PCT patent application WO 01/60804.

In general, the rosuvastatin methyl ammonium salt may be treated with an acid at a pH of about 1 to 5 to get rosuvastatin lactone. The reaction may be carried out in presence of a suitable solvent at a temperature of about −10 to 100° C. After completion of the reaction, the layers may be separated and organic layer after washing with water and/or brine may be concentrated completely under vacuum. The residue may be taken up in a second organic solvent. The mixture can be stirred at a temperature of from about 40 to about 150° C. for about 1 to 50 hours to affect lactonization. After completion of lactonization, the second organic solvent can be removed from the reaction mass under vacuum and the residue can be treated with third organic solvent to get the rosuvastatin lactone. The residue can be as such taken in the next step without actually isolating the lactone.

The acid may include inorganic acids such as hydrochloric acid, sulfuric acid, nitric acid, phosphoric acid, or mixtures thereof or organic acids such as formic acid, acetic acid, and the like.

Suitable solvents for lactonization reaction are solvents which are water immiscible or partially miscible. Examples of such solvents include toluene, xylene, benzene, ethyl methyl ketone, diisobutyl ketone, methyl isobutyl ketone, methyl t-butyl ether, diisopropyl ether, ethyl acetate, methyl formate, methyl acetate, isobutyl acetate, n-propyl acetate, isopropyl acetate, amyl acetate, or mixtures thereof.

Second organic solvents which may be used include methyl t-butyl ether, toluene, xylene, benzene, diisopropyl ether, n-butanol, isobutyl acetate, ethyl methyl ketone, diisobutyl ketone, or mixtures thereof.

Third organic solvents which may be added include solvents in which rosuvastatin is insoluble or very slightly soluble or sparingly soluble. Examples of such solvents include isopropanol, isobutanol, n-butanol, cyclopentane, cyclohexane, cycloheptane, hexane, petroleum ether, heptane, diethyl ether, diisopropyl ether, or mixtures thereof.

The lactone of Formula III may be dissolved in a solvent and treated with a base at a temperature of from about 10 to 70° C. for about, 1 to 40 hours to effect hydrolysis of the lactone. The pH of the reaction mass during the reaction can be adjusted in the range of about 7.5 to 11, using a base. The solvent may then be removed and the residue can be taken up in water. The aqueous solution may be washed with a solvent and then treated with calcium ions to obtain rosuvastatin calcium in amorphous form.

The base may include one or more of sodium hydroxide, sodium carbonate, sodium bicarbonate, potassium hydroxide, potassium carbonate, and potassium bicarbonate.

The calcium ions can be generated by using a calcium compound including, for example calcium chloride, calcium hydroxide, calcium carbonate, calcium acetate, calcium sulphate, calcium borate, calcium tartarate, calcium bromide, or any other compound capable of generating calcium ions.

The inventors also have developed a process for the preparation of pure amorphous rosuvastatin calcium, by treating rosuvastatin methyl ammonium salt with a base and a calcium salt; and isolating the amorphous rosuvastatin calcium.

The rosuvastatin methyl ammonium salt may be treated with a base in presence of water. It may also include an organic solvent. The reaction temperature can be kept at about −5 to 100° C.

The bases and calcium salts as described earlier may be used.

The organic solvent may include one or more of lower alkanols, ethers, esters, ketones, polar aprotic solvents, alkyl or cycloalkyl hydrocarbons, or mixtures thereof. Examples of alkanol include those primary, secondary and tertiary alcohols having from one to six carbon atoms. Suitable lower alkanol solvents include methanol, ethanol, n-propanol, isopropanol, isobutanol, and n-butanol. Examples of ketones include solvents such as acetone, ethyl methyl ketone, methyl isobutyl ketone and diisobutyl ketone. Examples of esters include solvents such as ethyl formate, methyl acetate, ethyl acetate, isopropyl acetate, n-propyl acetate, isobutyl acetate, butyl acetate and amyl acetate. Examples of ethers include tetrahydrofuran, 1,4-dioxane, diethyl ether and diisopropyl ether. A suitable polar aprotic solvent includes one or more of N,N-dimethylformamide, N,N-dimethylacetamide, dimethylsulphoxide, acetonitrile and N-methylpyrrolidone. Examples of alkyl or cycloalkyl hydrocarbons include one or more of cyclopentane, cyclohexane, cycloheptane, hexane, petroleum ether, and heptane. Mixtures of all of these solvents are also contemplated.

The inventors also have developed a process for the preparation of pure amorphous rosuvastatin calcium, by lactonizing the rosuvastatin methyl ammonium salt of Formula II, to obtain rosuvastatin lactone of Formula III; reacting the lactone form of rosuvastatin with a base and a calcium salt; removing water from reaction mass by azeotropic distillation to obtain a solution containing rosuvastatin calcium; and recovering the amorphous form of rosuvastatin calcium by removing solvent from the resultant solution.

In general, a suitable organic solvent capable of azeotropically removing water may be added. Alternatively, after treatment with calcium ions, an organic solvent which may be capable of dissolving rosuvastatin calcium and is immiscible or partially miscible with water may be added to the reaction mass. The solvent can be made immiscible in water by adding sodium chloride or calcium chloride to the aqueous layer. The layers may be separated and the organic layer containing rosuvastatin calcium may be dried over, for example calcium chloride, sodium sulphate, or molecular sieves, to remove traces of water. The organic layer may then be concentrated to remove solvent to get the desired amorphous rosuvastatin calcium.

The organic solvents which can be used include one or more of tetrahydrofuran, 1,4-dioxane, toluene, xylene, dichloromethane, ethyl formate, methyl acetate, ethyl acetate, isopropyl acetate, n-propyl acetate, isobutyl acetate, butyl acetate, amyl acetate, ethyl methyl ketone, methyl isobutyl ketone and diisobutyl ketone, or mixtures thereof.

The inventors have also developed a process for the preparation of pure amorphous rosuvastatin calcium, by treating rosuvastatin calcium with an acid to obtain rosuvastatin of Formula IV, and converting the rosuvastatin to the amorphous form of rosuvastatin calcium by treatment with a base and calcium salt.

The reaction can be carried out in presence of water. Additionally, it may also contain an organic solvent.

The acids which may be used include inorganic acids such as hydrochloric acid, sulphuric acid, phosphoric acid, hydrobromic acid, nitric acid, and the like or a mixture thereof or organic acids, for example formic acid, acetic acid, propionic acid, anhydrides of carboxylic acids, methanesulphonic acid, 4-toluenesulphonic acid, and the like.

The organic solvent may include one or more of lower alkanols, ethers, esters, ketones, polar aprotic solvents, alkyl or cycloalkyl hydrocarbons or mixtures thereof. Examples of alkanol include those primary, secondary and tertiary alcohols having from one to six carbon atoms. Suitable lower alkanol solvents include methanol, ethanol, n-propanol, isopropanol, isobutanol, and n-butanol. Examples of ketones include solvents such as acetone, ethyl methyl ketone, methyl isobutyl ketone and diisobutyl ketone. Examples of esters include solvents such as ethyl formate, methyl acetate, ethyl acetate, isopropyl acetate, n-propyl acetate, isobutyl acetate, butyl acetate and amyl acetate. Examples of ethers include tetrahydrofuran, 1,4-dioxane, diethyl ether and diisopropyl ether. A suitable polar aprotic solvent includes one or more of N,N-dimethylformamide, N,N-diethylacetamide, dimethylsulphoxide, acetonitrile and N-methylpyrrolidone. Examples of alkyl or cycloalkyl hydrocarbons include one or more of cyclopentane, cyclohexane, cycloheptane, hexane, petroleum ether, and heptane. Mixtures of all of these solvents are also contemplated.

The reaction mass can be concentrated to remove organic solvent and rosuvastatin can be isolated. Alternatively, the reaction mass may be treated with a base and calcium salt to get the amorphous form of rosuvastatin calcium. The bases and calcium salts as described earlier may be used.

The resulting pure amorphous form of rosuvastatin calcium may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, and ophthalmic) administration. The oral dosage forms may include solid dosage forms, like powder, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

The amorphous form of rosuvastatin calcium can be administered for the treatment of hyperlipidemia, hypercholesterolemia, and atherosclerosis, in a warm-blooded animal.

For the purpose of this disclosure, a warm-blooded animal is a member of the animal kingdom possessed of a homeostatic mechanism and includes mammals and birds.

The present invention is further illustrated by the following examples which are provided merely to be exemplary of the invention and do not limit the scope of the invention. Certain modifications and equivalents will be apparent to those skilled in the art and are intended to be included within the scope of the present invention.

### EXAMPLE 1

### Preparation of Amorphous Rosuvastatin Calcium

Step a) Preparation of Crystalline Rosuvastatin Calcium

Amorphous rosuvastatin calcium (5.0 gm) was added to a mixture of water (50 ml) and acetonitrile (50 ml) at 15° C. The mixture was warmed to 40° C. to obtain a solution. The solution was then cooled slowly to 25-30° C. and stirred for 16 hours. The crystalline product was separated by filtration at ambient temperature and dried at 50° C. under vacuum to give rosuvastatin calcium as white crystals.

Yield: 3.4 gm (68%)

Step b) Conversion of Crystalline Rosuvastatin Calcium to Amorphous Form

Crystalline rosuvastatin calcium (4.0 gm) was dissolved in tetrahydrofuran (12.0 ml) at about 25-30° C. The solution was filtered through celite bed and the bed was washed with tetrahydrofuran (2.0 ml). The clear filtrate and the washings were mixed and poured slowly into cyclohexane (120 ml) over 30 minutes at 25-30° C. under vigorous stirring. The resulting mixture was stirred at 25-30° C. for further 2.0 hours. The precipitated product was filtered and dried at 45° C. under vacuum to give amorphous rosuvastatin calcium as white product.

Yield: 3.05 gm (76%) (XRD as per FIG. 1 showed it to be an amorphous material)

HPLC Purity: 99.72%

### EXAMPLE 2

### Preparation of Amorphous Rosuvastatin Calcium

Crystalline rosuvastatin calcium (5.0 gm) was dissolved in tetrahydrofuran (15.0 ml) at about 25-30° C. The solution was filtered through celite bed and the bed was washed with tetrahydrofuran (2.0 ml). The clear filtrate and washings were mixed and poured slowly into n-hexane (150 ml) over 30 minutes at 25-30° C. under vigorous stirring.

The resulting mixture was stirred at 25-30° C. for further 3.0 hours. The precipitated product was filtered and dried at 45° C. under vacuum to give amorphous rosuvastatin calcium as white product.

Yield: 3.6 gm (72%)

### EXAMPLE 3

### Preparation of Amorphous Rosuvastatin Calcium

Crystalline rosuvastatin calcium (5.0 gm) was dissolved in tetrahydrofuran (15.0 ml) at about 25-30° C. The solution was filtered through celite bed and the bed was washed with tetrahydrofuran (2.0 ml). The clear filtrate and the washings were mixed and poured slowly into heptane (120 ml) over 30 minutes at 25-30° C. under vigorous stirring. The resulting mixture was stirred at 25-30° C. for further 3.0 hours. The precipitated product was filtered and dried at 45° C. under vacuum (about 5 to 10 mm of Hg) to give amorphous rosuvastatin calcium as white product.

Yield: 3.2 gm (64%)

### EXAMPLE 4

### Preparation of Amorphous Rosuvastatin Calcium

Crystalline rosuvastatin calcium (1.0 gm) was dissolved in tetrahydrofuran (3.0 ml) at about 25-30° C. The solution was filtered through celite bed and the bed was washed with tetrahydrofuran (0.5 ml). The clear filtrate and the washings were mixed and poured slowly into diethyl ether (25 ml) over 30 minutes at 20° C. under vigorous stirring. The resulting mixture was stirred at 20° C. for further 1.0 hours. The precipitated product was filtered and dried at 45° C. under vacuum (about 5 to 10 mm of Hg) to give amorphous rosuvastatin calcium as white product.

Yield: 0.80 gm (80%)

HPLC Purity: 99.62%

### EXAMPLE 5

### Preparation of Amorphous Rosuvastatin Calcium

Crystalline rosuvastatin calcium (1.0 gm) was dissolved in tetrahydrofuran (3.0 ml) at about 25-30° C. The solution was filtered through celite bed and the bed was washed with tetrahydrofuran (0.5 ml). The clear filtrate and the washings were mixed and poured slowly into isopropyl alcohol (25 ml) over 30 minutes at 20° C. under vigorous stirring. The resulting mixture was stirred at 20° C. for further 1.0 hours. The precipitated product was filtered and dried at 45° C. under vacuum (about 5 to 10 mm of Hg) to give amorphous rosuvastatin calcium as white product.

Yield: 0.6 gm (60%)

### EXAMPLE 6

### Preparation of Amorphous Rosuvastatin Calcium

Crystalline rosuvastatin calcium (2.0 gm) was dissolved in tetrahydrofuran (6.0 ml) at about 25-30° C. The solution was filtered through celite bed and the bed was washed with tetrahydrofuran (0.5 ml). The clear filtrate and the washings were mixed and poured slowly into isopropyl acetate (60.0 ml) over 20 minutes at 25-30° C. under vigorous stirring. The resulting mixture was stirred at 25-30° C. for further 5 minutes. The precipitated product was filtered immediately and dried at 45° C. under vacuum (about 5 to 10 mm of Hg) to give amorphous rosuvastatin calcium as white product.

Yield: 0.7 gm (35%)

### EXAMPLE 7

### Preparation of Amorphous Rosuvastatin Calcium

Crystalline rosuvastatin calcium (2.0 gm) was dissolved in dimethylsulphoxide (6.0 ml) at about 30-35° C. The solution was filtered through celite bed and the bed. The clear filtrate was poured slowly into water (15.0 ml) over 30 minutes at 25-30° C. under vigorous stirring. The resulting mixture was stirred at 25-30° C. for further 2.0 hours. The precipitated product was filtered and dried at 45° C. under vacuum (about 5 to 10 mm of Hg) to give amorphous rosuvastatin calcium as white product.

Yield: 1.5 gm (75%)

### EXAMPLE 8

### Preparation of Amorphous Rosuvastatin Calcium

Crystalline rosuvastatin calcium (5.0 gm) was dissolved in tetrahydrofuran (25.0 ml) at about 25-30° C. The solution was filtered through celite bed and the bed was washed with tetrahydrofuran (2.0 ml). The clear solution was spray dried at 25-30⁰C, 600 Newton litre per hour nitrogen flow and at a rate of about 2.5 ml per minute. The material was recovered from receiver and dried at 40-45⁰C under vacuum (about 5 to 10 mm of Hg) for 6 hrs to get the amorphous rosuvastatin calcium.

Yield: 4.0 gm (80%)

### EXAMPLE 9

### Preparation of Amorphous Rosuvastatin Calcium

Crystalline rosuvastatin calcium (100 gm) was dissolved in methylene chloride (500 ml) at about 25-30° C. The solution was filtered through celite bed and the bed was washed with methylene chloride (40 ml). The clear solution was spray dried at 38-40⁰C, 600 Newton litre per hour nitrogen flow and at a rate of about 15 to 20 ml per minute. The material was recovered from receiver and dried at 40-45⁰C under vacuum (about 5 to 10 mm of Hg) for 6 hrs to get the amorphous rosuvastatin calcium.

Yield: 85 gm (85%)

### EXAMPLE 10

### Preparation of Amorphous Rosuvastatin Calcium

Crystalline rosuvastatin calcium (1.0 gm) was dissolved in tetrahydrofuran (6.0 ml) at about 25-30° C. The solution was filtered through celite bed and the bed was washed with tetrahydrofuran (0.5 ml). The clear solution was concentrated under vacuum at 45⁰C to get solids which were then dried at 40-45⁰C under vacuum (about 5 to 10 mm of Hg) for 6 hrs to get the amorphous rosuvastatin calcium.

Yield: 0.9 gm (90%)

HPLC Purity: 99.71%

### EXAMPLE 11

### Preparation of Amorphous Rosuvastatin Calcium

Crystalline rosuvastatin calcium (2.0 gm) was slurried in cyclohexane (10 ml) and the slurry was placed in a glass mortar. The slurry was triturated with pestle till the crystalline form was completely converted to amorphous form. The slurry was then filtered and the solid was dried under vacuum at 40-45° C. to get amorphous rosuvastatin calcium.

Yield: 1.3 gm (65%)

### EXAMPLE 12

### Preparation of Amorphous Rosuvastatin Calcium

Crystalline rosuvastatin calcium (2.0 gm) was subjected to grinding using an agate pestle and mortar till it is completely converted to the amorphous form.

Yield: 1.70 gm (85%)

### EXAMPLE 13

### Preparation of Amorphous Rosuvastatin Calcium

Crystalline rosuvastatin calcium (1.0 gm) was dissolved in 1,4-dioxane (5.0 ml) at about 30-35° C. The clear solution was freeze-dried at a temperature of −20⁰C to get solids which were then dried at −20 to 10⁰C under vacuum (less than 0.1 mm of Hg) for 3 hrs to get the amorphous rosuvastatin calcium.

Yield: 0.98 gm (98%)

### EXAMPLE 14

### Preparation of Amorphous Rosuvastatin Calcium

Step a) Preparation of Rosuvastatin Lactone from Rosuvastatin Methyl Ammonium Salt

Rosuvastatin methyl ammonium salt (20 gm) was added into a mixture of ethyl acetate (100 ml) and water (200 ml) at 25-30° C. and the pH of the reaction mass was adjusted to about 3.0 with 6N hydrochloric acid. The layers were separated and the organic layer was washed with water (50 ml). The organic layer was concentrated under vacuum to get an oily crude product which was mixed with toluene (50 ml). The reaction mass was refluxed for about 6 hours and the solvent was removed under vacuum at 60° C. The residue obtained was stirred with hexane (100 ml) and the separated solid was filtered. The product was dried under vacuum till constant weight at 40-45° C. to get rosuvastatin lactone.

Step b) Conversion of Rosuvastatin Lactone to Amorphous Rosuvastatin Calcium

Rosuvastatin lactone as obtained in step a) was dissolved in methanol (100 ml) and water (100 ml). To this solution, 8% sodium hydroxide solution was added till the pH of the reaction mass was about 8.5 to 8.7 and stirred for further 3 hours. After ensuring the absence of rosuvastatin lactone by TLC, the solvent was removed under vacuum and the aqueous layer was washed with methyl tert-butyl ether (80 ml). The traces of methyl tert-butyl ether were removed under vacuum and to the aqueous layer, a solution of calcium chloride dihydrate (4.5 gm) in water (25 ml) was added at 20-22° C. with vigorous stirring. After complete addition, the mixture was stirred for further 2 hours at 20-22° C. and filtered, washed the cake with water (20 ml) thrice and then dried at 45° C. under vacuum to get the amorphous rosuvastatin calcium.

Yield: 15.3 gm (83%)

### EXAMPLE 15

### Preparation of Amorphous Rosuvastatin Calcium

Rosuvastatin lactone as obtained in step a) of Example 13 was dissolved in methanol (100 ml) and water (100 ml). To this solution, 8% sodium hydroxide solution was added till the pH of the reaction mass was about 8.5 to 8.7 and stirred for further 3 hours. After ensuring the absence of rosuvastatin lactone by TLC, the solvent was removed under vacuum and the aqueous layer was washed with methyl tert-butyl ether (80 ml). The traces of methyl tert-butyl ether were removed under vacuum and to the aqueous layer, a solution of calcium acetate (4.0 gm) in water (25 ml) was added at 20-22° C. with vigorous stirring. After complete addition, the mixture was stirred for further 2 hours at 20-22° C. and filtered, washed the cake with water (20 ml) thrice and then dried at 45° C. under vacuum to get the amorphous rosuvastatin calcium.

Yield: 13.8 gm (75%)

### EXAMPLE 16

### Preparation of Amorphous Rosuvastatin Calcium

Rosuvastatin methyl ammonium salt (10 gm) was added in water (50 ml) and sodium hydroxide solution (8%, 9.0 ml) was added to it at 25-30° C. and stirred for 20 minutes. The solution was filtered through celite bed and the bed was washed with water (20 ml). From the resulting clear filtrate, water was removed (about 40 ml) by vacuum distillation at about 60° C. To the resulting solution, water (40 ml) and a solution of calcium acetate (2 gm) in water (10 ml) was added at 20-22° C. under vigorous stirring. Solid rosuvastatin calcium precipitates out from reaction mass. To the reaction mass, tetrahydrofuran (50 ml) was added and stirred for 10 minutes. Sodium chloride (2.0 gm) was added to the reaction mass and stirred for further 10 minutes. The layers were separated and the organic layer was dried over powdered molecular sieves (10 gm). The molecular sieves were removed by filtration and the resultant solution was distilled azeotropically to remove water. After complete removal of water, tetrahydrofuran (50 ml) was added and the solution was filtered through celite bed. The clear filtrate was then concentrated under vacuum to get amorphous form of rosuvastatin calcium which was dried at 45° C. under vacuum.

Yield: 7.6 gm

### EXAMPLE 17

### Preparation of Amorphous Rosuvastatin Calcium

Crystalline rosuvastatin calcium (10.0 g) was added into a mixture of ethyl acetate (100 ml) and water (100 ml) at room temperature. The pH of the resulting solution was adjusted to about 4.0 to 4.2 by adding dilute hydrochloric acid at 25° C. The layers were separated and the organic layer was washed with water. The solvent was concentrated under vacuum to get an oily residue.

The oily residue obtained above was dissolved in methanol (35 ml) and water (50 ml) at room temperature. The pH of the solution was adjusted with sodium hydroxide (8% solution in water) to about 8.5 to 9.0 and the resulting reaction mass was stirred for further 1 hour at room temperature. Methanol was removed under vacuum. The oily residue was reconstituted in water (50 ml) and to the aqueous solution a solution of calcium acetate (2.1 gm) in water (10 ml) was added at 20-22° C. with vigorous stirring. After complete addition, the mixture was stirred for further 2 hours at 20-22° C. and filtered, washed the cake with water (20 ml) thrice and then dried at 45° C. under vacuum to get the amorphous rosuvastatin calcium.

Yield: 7.50 gm (75%) (XRD as per FIG. 1 showed it to be an amorphous material)

HPLC Purity: 99.59%

Assay: 99.6% w/w

While the present invention has been described in terms of its specific embodiments, certain modifications and equivalents will be apparent to those skilled in the art and are included within the scope of the present invention.

1. Amorphous rosuvastatin calcium of Formula I having a purity of more than 99% with diastereomeric impurity less than 0.5% by HPLC.

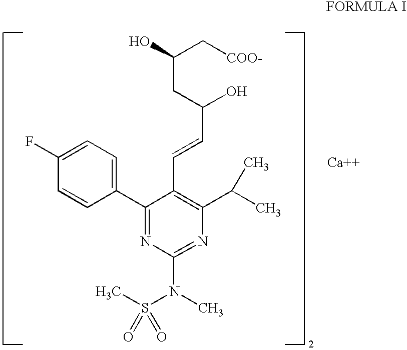

2. The amorphous form of rosuvastatin calcium of claim 1, wherein the rosuvastatin calcium has the X-ray diffraction pattern of FIG. 1.

3. A pharmaceutical composition comprising:
a therapeutically effective amount of an amorphous form of rosuvastatin calcium having purity greater than 99% with diastereomeric impurity less than 0.5% by HPLC; and one or more pharmaceutically acceptable carriers, excipients or diluents.

4. The pharmaceutical composition of claim 1, wherein the rosuvastatin calcium has the X-ray diffraction pattern of FIG. 1.

5. Amorphous rosuvastatin calcium having a purity of more than 99.5% with diastereomeric impurity less than 0.25% by HPLC.

6. The amorphous rosuvastatin calcium of claim 5 having a purity of more than 99.8% with diastereomeric impurity less than 0.15% by HPLC.

7. A process for the preparation of pure amorphous form of rosuvastatin calcium, the process comprising:
obtaining a solution of rosuvastatin calcium in one or more solvents; and
recovering the rosuvastatin calcium in the amorphous form from the solution thereof by the removal of the solvent.

8. The process of claim 7, wherein the solvent comprises one or more of lower alkanol, ketone, ether, ester, polar aprotic solvent, water, or mixtures thereof.

9. (canceled)

10. The process of claim 8, wherein the lower alkanol comprises one or more of methanol, ethanol, n-propanol, and isopropanol.

11. The process of claim 8, wherein the ketone comprises one or more of acetone, ethyl methyl ketone, methyl isobutyl ketone, and diisobutyl ketone.

12. The process of claim 8, wherein the ether comprises one or both of tetrahydrofuran, and 1,4-dioxane.

13. The process of claim 8, wherein the ester comprises one or more of ethyl formate, methyl acetate, ethyl acetate, isopropyl acetate, n-propyl acetate, isobutyl acetate, butyl acetate, and amyl acetate.

14. The process of claim 8, wherein the polar aprotic solvent comprises one or more of N,N-dimethylformamide, N,N-dimethylacetamide, dimethylsulphoxide, acetonitrile, and N-methylpyrrolidone.

15. The process of claim 7, wherein removing the solvent comprises one or more of distillation, distillation under vacuum, evaporation, spray drying, freeze-drying, lyophilization, filtration, filtration under vacuum, decantation, and centrifugation.

16. The process of claim 15 further comprising adding additional/second solvent before removing the solvent.

17. The process of claim 16, wherein the additional/second solvent comprises one or more of isopropanol, isobutanol, n-butanol, cyclopentane, cyclohexane, cycloheptane, hexane, petroleum ether, heptane, diethyl ether, diisopropyl ether, water, or mixtures thereof.

18. (canceled)

19. (canceled)

20. (canceled)

21. (canceled)

22. (canceled)

23. The process of claim 7, further comprising forming the product obtained into a finished dosage form.

24. (canceled)

25. A process for the preparation of pure amorphous form of rosuvastatin calcium, the process comprising:
subjecting crystalline rosuvastatin calcium to milling until said crystalline form is converted to the amorphous form.

26. The process of claim 25, wherein the crystalline form used is in solid state.

27. The process of claim 25, wherein slurry of the crystalline form in a solvent is used.

28. (canceled)

29. (canceled)

30. (canceled)

31. A process for the preparation of pure amorphous form of rosuvastatin calcium, the process comprising:
obtaining a solution of rosuvastatin calcium in one or more solvents; and
recovering the rosuvastatin calcium in the amorphous form from the solution thereof by freeze drying or lyophilizing.

32. The process of claim 31, wherein the solvent comprises one or more of lower alkanol, ketone, ether, ester, polar aprotic solvent, water, or mixtures thereof.

33. The process of claim 32, wherein the solvent comprises one or more of methanol, ethanol, isopropanol, n-propanol, tetrahydrofuran, 1,4-dioxane, ethyl formate, methyl acetate, ethyl acetate, isopropyl acetate, n-propyl acetate, isobutyl acetate, butyl acetate, amyl acetate, acetone, ethyl methyl ketone, methyl isobutyl ketone, diisobutyl ketone, N,N-dimethylformamide, N,N-dimethylacetamide, dimethylsulphoxide, acetonitrile, and N-methylpyrrolidone.

34. A process for the preparation of pure amorphous form of rosuvastatin calcium, the process comprising:
a) lactonizing rosuvastatin methyl ammonium salt of Formula II, 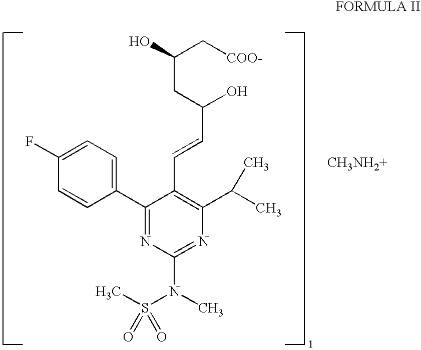
to obtain rosuvastatin lactone of Formula III, 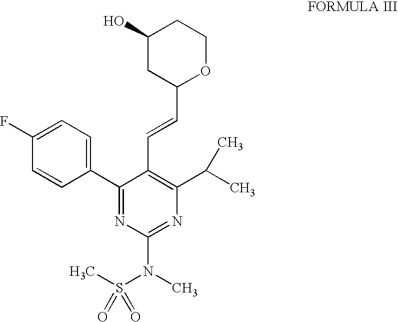
b) reacting the rosuvastatin lactone with a base and a calcium salt, and
c) recovering the amorphous form of rosuvastatin calcium.

35. The process of claim 34, wherein the lactonization is carried out in the presence of an acid in a solvent.

36. (canceled)

37. The process of claim 35, wherein the acid comprises one or more of hydrochloric acid, sulfuric acid, nitric acid, phosphoric acid, formic acid, acetic acid, or mixtures thereof.

38. The process of claim 35, wherein the solvent comprises one or more of toluene, xylene, benzene, ethyl methyl ketone, diisobutyl ketone, methyl isobutyl ketone, methyl t-butyl ether, diisopropyl ether, ethyl acetate, methyl formate, methyl acetate, isobutyl acetate, n-propyl acetate, isopropyl acetate, amyl acetate, or mixtures thereof.

39. The process of claim 34, wherein the rosuvastatin lactone is isolated.

40. The process of claim 34, wherein the base comprises one or more of sodium hydroxide, sodium carbonate, sodium bicarbonate, potassium hydroxide, potassium carbonate, and potassium bicarbonate.

41. The process of claim 34, wherein the calcium salt comprises one or more of calcium chloride, calcium hydroxide, calcium carbonate, calcium acetate, calcium sulphate, calcium borate, calcium tartarate, and calcium bromide.

42. (canceled)

43. (canceled)

44. A process for the preparation of pure amorphous form of rosuvastatin calcium, the process comprising:
treating rosuvastatin methyl ammonium salt with a base and a calcium salt; and
recovering the amorphous form of rosuvastatin calcium.

45. The process of claim 44, wherein the base comprises one or more of sodium hydroxide, sodium carbonate, sodium bicarbonate, potassium hydroxide, potassium carbonate, and potassium bicarbonate.

46. The process of claim 44, wherein the calcium salt comprises one or more of calcium chloride, calcium hydroxide, calcium carbonate, calcium acetate, calcium sulphate, calcium borate, calcium tartarate, and calcium bromide.

47. A process for the preparation of pure amorphous form of rosuvastatin calcium, the process comprising:
a) lactonizing rosuvastatin methyl ammonium salt of Formula II, 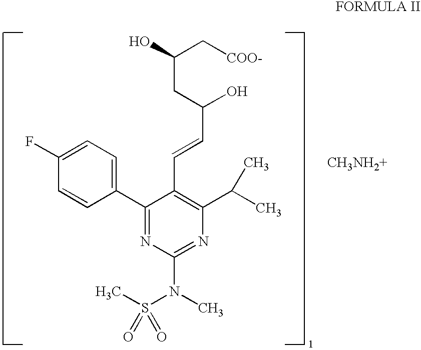
to obtain rosuvastatin lactone of Formula III, 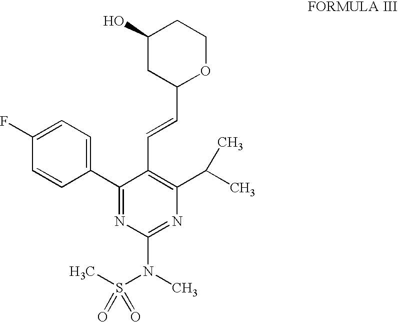
b) reacting the lactone form of rosuvastatin with a base and a calcium salt,
c) removing water from reaction mass by azeotropic distillation to obtain a solution containing rosuvastatin calcium, and
d) recovering the amorphous form of rosuvastatin calcium by removing solvent from the resultant solution.

48. A process of the preparation of pure amorphous form of rosuvastatin calcium, the process comprising:
a) treating rosuvastatin calcium with an acid to obtain rosuvastatin of
Formula IV, and 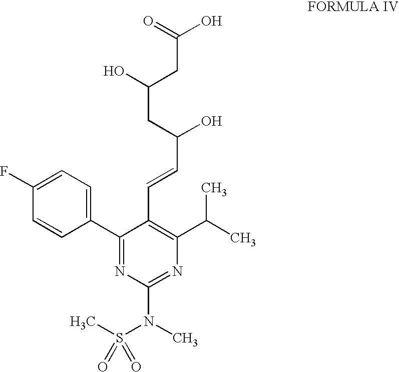
b) converting the rosuvastatin to the amorphous form of rosuvastatin calcium by treatment with a base and a calcium salt.

49. (canceled)

50. The process of claim 48, wherein the acid comprises one or more of hydrochloric acid, sulphuric acid, phosphoric acid, hydrobromic acid, nitric acid, formic acid, acetic acid, propionic acid, methanesulphonic acid, 4-toluenesulphonic acid, or mixtures thereof.

51. (canceled)

52. (canceled)

53. (canceled)

54. A method of treating hyperlipidemia, hypercholesterolemia, and atherosclerosis in a warm-blooded animal comprising administering a pharmaceutical composition that includes the pure amorphous form of rosuvastatin calcium having a purity of more than 99% with diastereomeric impurity less than 0.5% by HPLC.
